(19) United States
(12) Patent Application Publication
(10) Pub. No.: US 2002/0115216 A1
(43) Pub. Date: Aug. 22, 2002

(54) Composition for delivery of compounds to cells

(76) Inventors: Clifford J. Steer, St. Paul, MN (US); Betsy T. Kren, Minneapolis, MN (US); Cheryle Linehan-Stieers, Chaska, MN (US); R. Scott McIvor, St. Louis Park, MN (US); Perry B. Hackett, Shoreview, MN (US)

Correspondence Address: MUETING, RAASCH & GEBHARDT, P.A., P.O. BOX 581415, MINNEAPOLIS, MN, 55458, US

(21) Appl. No.: 09/861,014

(22) Filed: May 19, 2001

Related U.S. Application Data
(60) Provisional application No. 60/206,002, filed on May 19, 2000.
(60) Provisional application No. 60/285,121, filed on Apr. 20, 2001.

(51) Int. Cl. A61K 48/00; C07H 21/04; C12N 15/87

(52) U.S. Cl. 435/455; 536/23.1; 514/44; 525/54.2

(57) ABSTRACT

The present invention provides cationic polymers that include a primary amine and a targeting group covalently bound to the primary amine, wherein the targeting group targets a cell of interest by interacting with the surface of the cell. The invention also provides molecular complexes that include a polyethyleneimine and a targeting group covalently bound to a primary amine of the polyethyleneimine, and a biologically active compound. The invention further provides methods for delivering a biologically active compound to a vertebrate cell.

### CONTINUING APPLICATION DATA

This application claims the benefit of U.S. Provisional Application No. 60/206,002, filed May 19, 2000, and of U.S. Provisional Application No. 60/285,121, filed Apr. 20, 2001, which are both incorporated by reference herein.

### GOVERNMENT FUNDING

The present invention was made with government support under Grant No. P01-HD32652, awarded by the National Institute of Child Health and Human Development (NICHD). The Government has certain rights in this invention.

### BACKGROUND

In the early 1970s the first human genes were transferred into mammalian cells in the form of hybridomas. Since that time, scientists have been coercing nucleic acids into vertebrate cells. The introduction of nucleic acids into cells permits correcting a genetic deficiency or abnormality, for instance mutations, aberrant expression, and the like. The introduction of nucleic acids into cells can also be used to cause expression of a therapeutic protein in the affected cell or organ. This genetic information may be introduced either into a cell extracted from an organ, the modified cell then being reintroduced into the body, or directly in vivo into the appropriate tissue. Many advances have been made in the delivery of nucleic acids to cells, including the use of viral vectors and transfection techniques using cationic lipids and cations polymers to complex nucleic acids. However, there remains a need for methods to deliver nucleic acids to cells.

### SUMMARY OF THE INVENTION

The present invention represents an advance in the art of introducing biologically active compounds to cells. Cationic polymers have been used to complex polynucleotides, thereby protecting them from degradation before delivery of the polynucleotides to the nucleus, while simultaneously increasing their endocytic uptake into cells. The presence of free amino groups on cationic polymers makes them amenable to chemical modification for the attachment of ligands capable of targeting specific tissues. Polyethyleneimine (PEI), a cationic polymer used to complex polynucleotides, contains three free amino groups, a primary, a secondary, and a tertiary amine, and the secondary has been used as the site for the attachment of ligands to the PEI. As described herein, when lactose, a ligand capable of targeting liver cells, was added to the primary amino group of PEI, there was an unexpected and surprising increase in the rates of introducing complexed polynucleotides to cells. In addition, the covalent attachment of molecules to the primary amines of PEI also advantageously has less of an effect on secondary structure of the PEI during condensation of a PEI molecule and a complexed polynucleotide.

In one aspect, the invention provides a cationic polymer, preferably a polyethyleneimine, that contains a primary amine covalently bound to a targeting group. The cationic polymer is useful to deliver a compound to a cell, and the targeting group is thus one that is capable of targeting the cationic polymer to the cell of interest, preferably by interacting, directly or indirectly, with the surface of the cell. Preferably, the targeting group targets the cationic polymer to a liver cell, such as a hepatocyte. The targeting group is preferably a lactose.

In another aspect, the invention provides a molecular complex useful for delivery of a compound to a cell. In one embodiment, the molecular complex includes a cationic polymer, preferably a polyethyleneimine, that has a targeting group covalently bound to a primary amine; and a biologically active compound. The biologically active compound is preferably a polynucleotide.

In another embodiment, the molecular complex includes a cationic polymer, preferably a polyethyleneimine, that has a covalently bound targeting group; and a polynucleotide comprising a nucleic acid sequence flanked by inverted repeat sequences that bind a transposase. In this embodiment, the targeting group is preferably covalently bound to a primary amine of the cationic polymer although it can be covalently bound elsewhere on the cationic polymer, for example to a secondary or tertiary amine of the polymer. Optionally the molecular complex contains a second polynucleotide that includes a coding sequence encoding a transposase that binds to the inverted repeat sequences. Alternatively, the nucleic acid sequence flanked by the inverted repeat sequences and the coding sequence encoding a transposase can be present on the same polynucleotide. In yet another embodiment, the molecular complex can include as the biologically active compound only the coding sequence encoding a transposase. In that case, the inverted repeat sequences can, if desired, be delivered to the cell of interest by way of a second molecular complex.

In another aspect, the invention provides a method for making a cationic polymer:targeting group conjugate. One embodiment of the method encompasses converting a lactose to an aldonic acid, then combining the aldonic acid, a polyethyleneimine and 1-ethyl-3-(dimethylaminopropyl)-carbodiimide under conditions suitable for coupling the aldonic acid to primary amines of the polyethyleneimine to yield the cationic polymer:targeting group conjugate. Another embodiment of the method encompasses combining a lactose, a polyethyleneimine, and 1-ethyl-3-(dimethylaminopropyl)-carbodiimide under conditions suitable for coupling the lactose to primary amines of the polyethyleneimine to yield the cationic polymer:targeting group conjugate.

In yet another aspect, the invention provides a composition that includes the cationic polymer of the invention and a pharmaceutical carrier. Preferably the composition includes a molecular complex that contains a polyethyleneimine having a covalently bound targeting group; and a polynucleotide that contains a nucleic acid sequence flanked by inverted repeat sequences that bind a transposase and a coding sequence encoding a transposase that binds to the inverted repeat sequences. The nucleic acid sequence preferably comprises a coding sequence encoding bilirubin UDP-glucuronosyltransferase-1 (UGT1A1); the targeting group is preferably lactose; and the cell targeted by the targeting group is preferably a liver cell. Optionally, the molecular complex included in the composition contains a second polynucleotide that includes a coding sequence encoding a transposase that binds to the inverted repeat sequences. Alternatively, the nucleic acid sequence flanked by the inverted repeat sequences and the coding sequence encoding a transposase can be present on the same polynucleotide.

In yet another aspect, the invention provides a method for delivering a biologically active compound as described herein to a vertebrate cell. The method involves introducing into the vertebrate cell a molecular complex as described herein that includes the biologically active compound and a cationic polymer of the invention. The targeting molecule is preferably bound to a primary amine of the cationic polymer. In embodiments wherein the biological compound includes a polynucleotide containing a nucleic acid sequence flanked by inverted repeat sequences that bind a transposase and/or a coding sequence encoding a transposase that binds to the inverted repeat sequences, the targeting molecule can be bound to other locations on the cationic polymer, although binding to a primary amine on the cationic polymer remains preferred. The cationic polymer preferably includes a polyethyleneimine, more preferably a polyethyleneimine having a lactose covalently bound to a primary amine of the polyethyleneimine. In a particularly preferred embodiment, the molecular complex is delivered to a liver cell, preferably a hepatocyte, and the nucleic acid sequence flanked by the inverted repeat sequences includes a coding sequence encoding bilirubin UDP-glucuronosyltransferase-1 (UGT1A1). The method can be performed in vivo, ex vivo, or in utero.

In yet another aspect, the invention provides a method for delivering a biologically active compound to a vertebrate cell that includes introducing a naked polynucleotide into a vertebrate cell, wherein the naked polynucleotide comprises a nucleic acid sequence flanked by inverted repeat sequences that bind a transposase. In one embodiment, the vertebrate cell is in an in utero animal; in another embodiment, the vertebrate cell is in an animal.

Unless otherwise specified, “a,” “an,” “the,” and “at least one” are used interchangeably and mean one or more than one.

### BRIEF DESCRIPTION OF THE FIGURES

FIG. 1(A) is a double-stranded nucleic acid sequence encoding the SB protein (SEQ ID NO: 10). FIG. 1(B) is the amino acid sequence (SEQ ID NO: 9) of an SB transposase. The major functional domains are highlighted; NLS, a bipartite nuclear localization signal; the boxes marked D and E comprising the DDE domain (Doak, et al., Proc. Natl. Acad, Sci., USA, 91, 942-946 (1994)) that catalyzes transposition; DD(34)E box, a catalytic domain containing two invariable aspartic acid residues, D(153) and D(244), and a glutaric acid residue, E(279), the latter two separated by 43 amino acids.

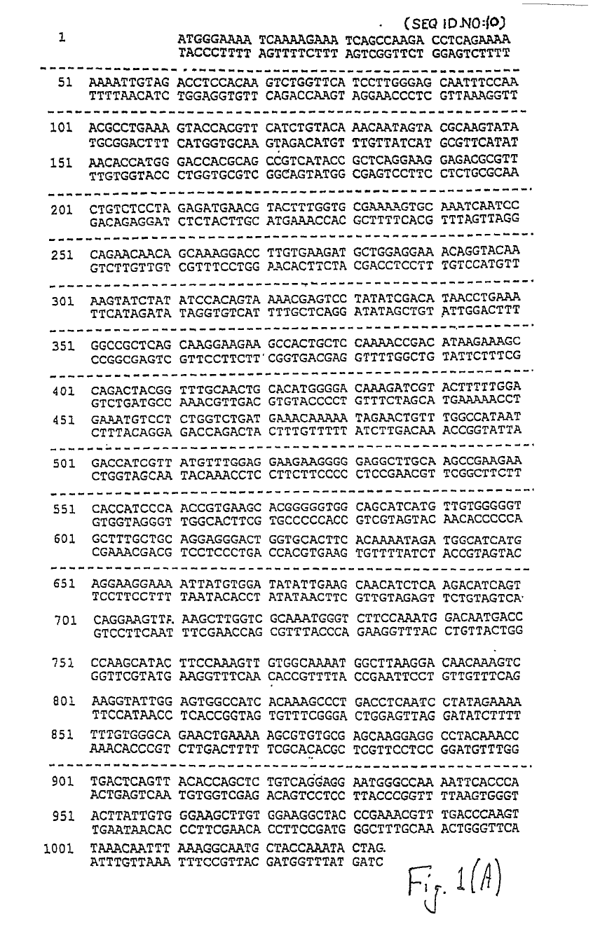

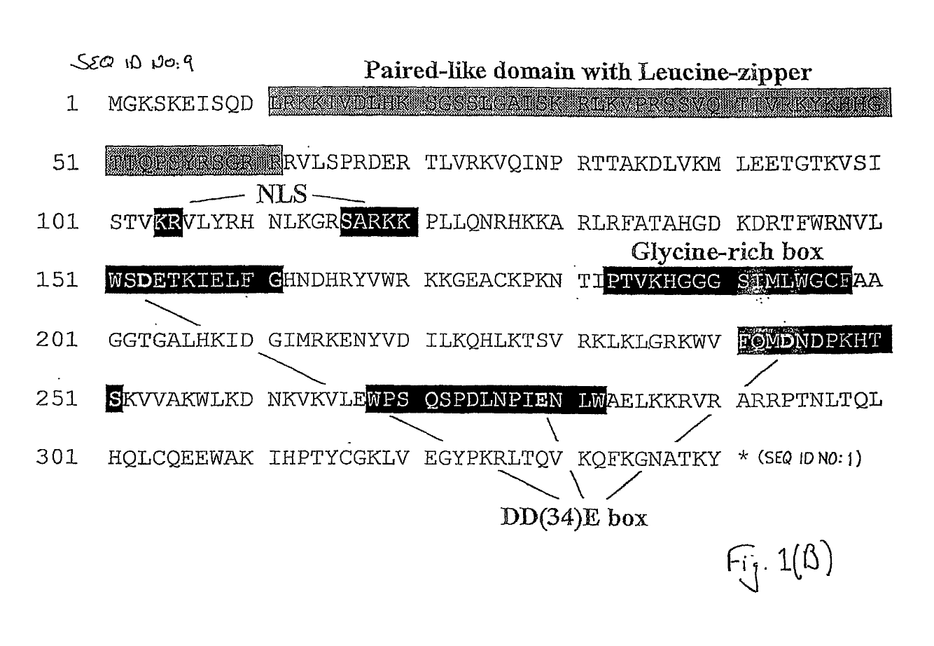

### DETAILED DESCRIPTION OF PREFERRED EMBODIMENTS OF THE INVENTION

Compositions

The present invention provides cationic polymers that include a targeting group covalently bound to an amine, preferably a primary amine, of the cationic polymer. A cationic polymer that includes a targeting group covalently bound to the cationic polymer is sometimes referred to herein as a “cationic polymer:target molecule conjugate” or a “cationic polymer conjugate.” As used herein, a “cationic polymer” is a polymer with an net positive charge at physiological pH. Examples of cationic polymers include polylysine and polyarginine. A preferred cationic polymer is polyethyleneimine (PEI). The PEI useful in the present invention can be linear or branched, preferably branched. One example of a branched PEI has the structure:

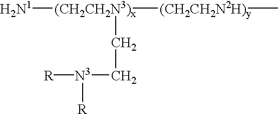

where N¹ refers to the primary amine, N² refers to the secondary amine, and N³ refers to the tertiary amine, R is either a single ethyleneimine (CH₂CH₂NH₂) or a polyethyleneimine (CH₂CH₂NH₂)ₓ, and x and y are each independently integers that are greater than one (see, for instance, Klotz et al., Biochem., 8, 4752-4756 (1969)). Other PEI polymers are known in the art and can be conjugated in accordance with the invention. The cationic polymers, preferably PEI, can be obtained commercially, for instance from Sigma-Aldrich (St. Louis, Mo.).

As used herein, a “targeting group” and “targeting molecule” are used interchangeably, and refer to a chemical species that interacts, either directly or indirectly, with the surface of a cell, for instance with a molecule present on the surface of a cell, e.g., a receptor. The interaction can be, for instance, an ionic bond, a hydrogen bond, a Van der Waals force, or a combination thereof. Examples of targeting groups include, for instance, saccharides, polypeptides (including hormones), polynucleotides, fatty acids, and catecholamines. As used herein, the term “saccharide” refers to a single carbohydrate monomer, for instance glucose, or two or more covalently bound carbohydrate monomers, i.e., an oligosaccharide. An oligosaccharide including 4 or more carbohydrate monomers can be linear or branched. Examples of oligosaccharides include lactose, maltose, and mannose. As used herein, “polypeptide” refers to a polymer of amino acids and does not refer to a specific length of a polymer of amino acids. Thus, for example, the terms peptide, oligopeptide, protein, antibody, and enzyme are included within the definition of polypeptide. This term also includes post-expression modifications of the polypeptide, for example, glycosylations (e.g., the addition of a saccharide), acetylations, phosphorylations and the like.

Preferably, the interaction between the targeting group and a molecule present on the surface of a cell, e.g., a receptor, results in the uptake of the targeting group by the cell (as well as the covalently attached cationic polymer and a complexed biologically active compound), for instance by endocytosis. Preferably, the receptor is endocytosed through clathrin-coated pits to endosomes. In those aspects of the invention where the targeting group is used to deliver the PEI to liver cells, preferably a hepatocyte, examples of such receptors include the low density lipoprotein receptor and the asialoglycoprotein receptor. Preferred examples of targeting groups include galactose, N-acetylgalactosamine, triantennary galactose, linear tetra galactose, lactose, and asialofeutin, each of which interacts with the asialoglycoprotein receptor. Other examples of targeting groups include, for instance, antibodies that bind to a molecule present on the surface of a cell, preferably a receptor.

Optionally and preferably, the cationic polymer of the present invention, preferably PEI, in complexed with a biologically active compound. As used herein, the term “biologically active compound” includes molecules having a net negative charge at physiological pH. Examples of compounds that can be used herein include, for instance, polynucleotides and polypeptides, and combinations thereof. As used herein, “biologically active compounds” include compounds that are able to modify a cell in any way, including modifying the metabolism of the cell, and also include compounds that permit the cell containing the molecule to be detected. As used herein, the term “polynucleotide” refers to a polymeric form of nucleotides of any length, either ribonucleotides or deoxynucleotides, and includes both double—and single-stranded DNA and RNA, and combinations thereof. A polynucleotide may include nucleotide sequences having different functions, including for instance coding sequences, and non-coding sequences such as regulatory sequences. Coding sequence, non-coding sequence, and regulatory sequence are defined below. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, or a fragment. Preferably, a polynucleotide complexed with a cationic polymer of the invention includes a coding sequence.

A “coding sequence” or a “coding region” is a polynucleotide that encodes a polypeptide and, when placed under the control of appropriate regulatory sequences expresses the encoded polypeptide. The boundaries of a coding region are generally determined by a translation start codon at its 5′ end and a translation stop codon at its 3′ end. A regulatory sequence is a nucleotide sequence that regulates expression of a coding region to which it is operably linked. Nonlimiting examples of regulatory sequences include promoters, transcription initiation sites, translation start sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

Typically, a biologically active compound complexed with a cationic polymer, preferably PEI, is a molecule that modifies in some way the cell to which it is delivered. For instance, a molecule may modify the expression of an endogenous coding sequence or the activity of a polypeptide encoded by an endogenous coding sequence. In an aspect of the invention, a polynucleotide may be used to alter the nucleotide sequence of a polynucleotide present in a cell (e.g., in the cell's genomic DNA). Such polynucleotides may alter one or more nucleotides in a regulatory region, and result in modified expression (for instance, increased or decreased expression) of an operably linked coding sequence, or such polynucleotides may alter one or more nucleotides in a coding sequence present in a cell, and modify the activity of a polypeptide encoded by the coding sequence. Examples of polynucleotides that can be used to alter the nucleotide sequence of a polynucleotide present in a cell include polynucleotides that have a contiguous stretch of RNA and DNA nucleotides in a duplex conformation (see, for instance, Bandyopadhyay et al., J. Biol. Chem., 274, 10163-101172 (1990)). Other types of polynucleotides that can be complexed with a cationic polymer and modify expression of an endogenous coding sequence include, for instance, an antisense RNA or a double stranded RNA.

A biologically active compound complexed with a cationic polymer may result in the presence of an exogenous polypeptide in the cell to which the biologically active compound is introduced. For instance, the biologically active compound may be a polynucleotide that includes an exogenous coding sequence. “Exogenous coding sequence” refers to a foreign coding region, i.e., a coding region that is not normally present in the cell to which it is introduced. Exogenous coding sequences include those that can be used to correct a genetic deficiency. An example of an exogenous coding sequence encoding an exogenous polypeptide is the UDP-glucuronosyltransferase-1 polypeptide, which is able to correct a genetic deficiency in the coding sequence encoding UDP-glucuronosyltransferase-1, the UGT1A1 gene. Alternatively, the biologically active compound may be the exogenous polypeptide that is active in the cell. For instance, an exogenous coding sequence may encode a marker. Markers and marker sequences are defined herein. In another aspect of the invention, a polynucleotide complexed to a cationic polymer may be catalytic. Examples of catalytic polynucleotides include, for instance, catalytic RNAs.

Biologically active compounds delivered to a cell may be therapeutic (i.e., able to treat or prevent a disease) or non-therapeutic (i.e., not directed to the treatment or prevention of a disease). Examples of diseases that can be treated or prevented with therapeutic biologically active compounds include, for instance, liver specific diseases such as hemophilia A, hemophilia B, Crigler-Najjar syndrome Type I, and ornithine transcarbamylase deficiency. Non-therapeutic biologically active compounds include detection or diagnostic compounds, including markers, that can be used in, for instance, detecting the presence of a particular cell, distinguishing cells, detecting whether a targeting group is functioning to target a particular tissue, and/or whether the transposons disclosed herein function when delivered to cells using the compositions of the present invention.

A polynucleotide complexed to a cationic polymer may be a portion of a vector. A vector is a replicating polynucleotide, such as a plasmid, viral, or cosmid, to which another polynucleotide may be attached so as to bring about the replication of the attached polynucleotide. The vector may include a coding sequence. A vector can provide for further cloning (amplification of the polynucleotide), i.e., a cloning vector, or for expression of the polypeptide encoded by the coding region, i.e., an expression vector. Preferably, a vector useful in the present invention is an expression vector. The term vector includes, but is not limited to, plasmid vectors, viral vectors, cosmid vectors, or artificial chromosome vectors. Examples of viral vectors include adenovirus, herpes simplex virus (HSV), alphavirus, simian virus 40, picomavirus, vaccinia virus, and adeno-associated virus. Preferably the vector is a plasmid. In some aspects of the invention, a vector is capable of replication in the cell to which it is introduced; in other aspects the vector is not capable of replication.

Selection of a vector depends upon a variety of desired characteristics in the resulting construct, such as a selection marker, vector replication rate, and the like. An expression vector optionally includes regulatory sequences operably linked to the coding sequence such that the coding region is expressed in the cell. The invention is not limited by the use of any particular promoter, and a wide variety are known. Promoters act as regulatory signals that bind RNA polymerase in a cell to initiate transcription of a downstream (3′ direction) operably linked coding sequence. The promoter used in the invention can be a constitutive or an inducible promoter. It can be, but need not be, heterologous with respect to the cell to which it is introduced.

An expression vector can optionally include a ribosome binding site (a Shine Dalgarno site for prokaryotic systems or a Kozak site for eukaryotic systems) and a start site (e.g., the codon ATG) to initiate translation of the transcribed message to produce the encoded polypeptide. It can also include a termination sequence to end translation. A termination sequence is typically a codon for which there exists no corresponding aminoacetyl-tRNA, thus ending polypeptide synthesis. The polynucleotide used to transform the host cell can optionally further include a transcription termination sequence.

The vector optionally includes one or more marker sequences, which typically encode a marker that can be detected. A marker sequence includes, for instance, a fluorescent marker. Examples of fluorescent markers include green fluorescent protein, blue fluorescent protein, and red fluorescent protein.

In a preferred aspect of the invention, a vector includes a transposon element, also referred to herein as a “transposon.” A transposon includes a polynucleotide that includes a nucleic acid sequence flanked by cis-acting nucleotide sequences on the termini of the transposon. The nucleic acid sequence flanked by the IRs can include a coding sequence and/or a non-coding sequence. The present invention is not limited to the use of a particular transposon element. Preferably, the transposon is able to excise from the vector and integrate into the cell's genomic DNA. A nucleic acid sequence is “flanked by” cis-acting nucleotide sequences if at least one cis-acting nucleotide sequence is positioned 5′ to the nucleic acid sequence, and at least one cis-acting nucleotide sequence is positioned 3′ to the nucleic acid sequence. Cis-acting nucleotide sequences include at least one inverted repeat (IR) at each end of the transposon, to which a transposase, preferably a member of the Sleeping Beauty (SB) family of transposases, binds. The SB family of transposases is described in greater detail below.

Each inverted repeat preferably includes one or more direct repeats. The nucleotide sequence of the direct repeat is preferably at least about 80% identical with a consensus direct repeat sequence (SEQ ID NO: 1) which is described below. A direct repeat is typically between about 25 and about 35 base pairs in length, preferably about 29 to about 31 base pairs in length. Notwithstanding the above, however, an inverted repeat optionally contains only one direct “repeat,” in which event the direct repeat is not actually a “repeat” but is nonetheless a polynucleotide having at least about 80% identity to a consensus direct repeat sequence as described more fully below.

In some aspects of the invention there are two direct repeats in each inverted repeat sequence. The direct repeats (which number, in this embodiment, four) have similar polynucleotides, as described in more detail below. An inverted repeat on the 5′ or “left” side of a transposon of this embodiment typically comprises a direct repeat (i.e., a left outer repeat), an intervening region, and a second direct repeat (i.e., a left inner repeat). An inverted repeat on the 3′ or “right” side of a transposon of this embodiment comprises a direct repeat (i.e., a right inner repeat), an intervening region, and a second direct repeat (i.e., a right outer repeat). The intervening region within an inverted repeat is generally at least about 150 base pairs in length, preferably at least about 160 base pairs in length. The intervening region is preferably no greater than about 200 base pairs in length, more preferably no greater than about 180 base pairs in length. The nucleotide sequence of the intervening region of one inverted repeat may or may not be similar to the nucleotide sequence of an intervening region in another inverted repeat.

Most transposons have perfect inverted repeats, whereas the inverted repeats that bind SB protein contain direct repeats that preferably have at least about 80% identity to a consensus direct repeat, preferably about 90%, more preferably about 95% identity to a consensus direct repeat. A preferred consensus direct repeat is 5′-CMSWKKRRGTCRGAAGTTTACATACACTTAAK (SEQ ID NO: 1) where M is A or C, S is G or C, W is A or T, K is G or T, and R is G or A. The presumed core binding site of SB protein is nucleotides 3 through 31 of SEQ ID NO: 1. Nucleotide identity is defined in the context of a comparison between a direct repeat and SEQ ID NO: 1, and is determined by aligning the residues of the two polynucteotides (i.e., the nucleotide sequence of the candidate direct repeat and the nucleotide sequence of SEQ ID NO: 1) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate direct repeat is the direct repeat being compared to SEQ ID NO: 1. Preferably, two nucleotide sequences are compared using the Blastn program of the BLAST 2 search algorithm, as described by Tatusova, et al. (FEMS Microbiol Lett, 174, 247-250 (1999)), and available at www.ncbi.nlm.nih.gov/gorf/bl2.html. Preferably, the default values for all BLAST 2 search parameters are used, including reward for match=1, penalty for mismatch =−2, open gap penalty=5, extension gap penalty=2, gap x_dropoff=50, expect=10, wordsize=11, and filter on. In the comparison of two nucleotide sequences using the BLAST search algorithm, nucleotide identity is referred to as “identities.”

Examples of direct repeat sequences that bind to SB protein include: a left outer repeat 5′-GTTGAAGTCGGAAGTTTACATACACTTAA-3′ (SEQ ID NO: 2); a left inner repeat 5′-CAGTGGGTCAGAAGTTTACATACACTAAG-3′ (SEQ ID NO:3); a right inner repeat 5′-CAGTGGGTCAGAAGTTAACATACACTCAATT-3′ (SEQ ID NO: 4); and a right outer repeat 5′

| | |
|---|---|
| 5′-AGTTGAAGTCGGAAGTTTACATACACCTTAG-3′ | (SEQ ID NO:5) |

In one embodiment the direct repeat sequence includes at least the following sequence: ACATACAC (SEQ ID NO: 6).

One preferred inverted repeat sequence
of this invention is SEQ ID NO:7

```
5′-AGTTGAAGTC GGAAGTTTAC ATACACTTAA GTTGGAGTCA TTAAAACTCG
      TTTTTCAACT ACACCACAAA TTTCTTGTTA ACAAACAATA GTTTTGGCAA
      GTCAGTTAGG ACATCTACTT TGTGCATGAC ACAAGTCATT TTTCCAACAA
      TTGTTTACAG ACAGATTATT TCACTTATAA TTCACTGTAT CACAATTCCA
      GTGGGTCAGA AGTTTACATA CACTAA-3′
```

and another preferred inverted repeat sequence
of this invention is SEQ ID NO:8

```
5′-TTGAGTGTAT GTTAACTTCT GACCCACTGG GAATGTGATG AAAGAAATAA
      AAGCTGAAAT GAATCATTCT CTCTACTATT ATTCTGATAT TTCACATTCT
      TAAAATAAAG TGGTGATCCT AACTGACCTT AAGACAGGGA ATCTTTACTC
      GGATTAAATG TCAGGAATTG TGAAAAAGTG AGTTTAAATG TATTTGGCTA
      AGGTGTATGT AAACTTCCGA CTTCAACTG-3′.
```

The inverted repeat (SEQ ID NO: 8) contains the poly(A) signal AATAAA at nucleotides 104-109. This poly(A) signal can be used by a coding sequence present in the transposon to result in addition of a poly(A) tail to an mRNA. The addition of a poly(A) tail to an MRNA typically results in increased stability of that MRNA relative to the same MRNA without the poly(A) tail.

In those aspects of the invention where a cationic polymer is complexed with a transposon, the cationic polymer is optionally also complexed with a transposase. The present invention is not limited to the use of a particular transposase, provided the transposase mediates the excision of a transposon from a vector and subsequent integration of the transposon into the genomic DNA of a target cell. The transposase may be present as a polypeptide that includes a coding sequence encoding a transposase. Alternatively and preferably, the transposase complexed is present as a polynucleotide. The polynucleotide can be RNA, for instance an MRNA encoding the transposase, or DNA, for instance a coding sequence encoding the transposase. When the transposase is present as a coding sequence encoding the transposase, in some aspects of the invention the coding sequence may be present on the same vector that includes the transposon. In other aspects of the invention, the transposase coding sequence may be present on a second vector which is also complexed with the cationic polymer.

A preferred transposase for use in the invention is “Sleeping Beauty” transposase, referred to herein as SB transposase or SB polypeptide (Z. Ivies et al. Cell, 91, 501-510 (1997); WO 98/40510 (Hackett et al.); WO 99/25817 (Hackett et al.)). SB transposase is able to bind the inverted repeat sequences of SEQ ID NOs:7-8 and direct repeat sequences (SEQ ID NOs:2-5) from a transposon, as well as a consensus direct repeat sequence (SEQ ID NO: 1). SB transposase includes, from the amino-terminus moving to the carboxy-terminus, a paired-like domain possibly with a leucine zipper, one or more nuclear localizing domains (NLS) domains and a catalytic domain including a DD(34)E box and a glycine-rich box, as described in WO 98/40510 (Hackett et al.). The SB family of polypeptides includes the polypeptide having the amino acid sequence of SEQ ID NO: 9. Preferably, a member of the SB family of polypeptides also includes polypeptides with an amino acid sequence that shares at least about 80% amino acid identity to SEQ ID NO: 9; more preferably, it shares at least about 90% amino acid identity therewith, most preferably, about 95% amino acid identity. Amino acid identity is defined in the context of a comparison between the member of the SB family of polypeptides and SEQ ID NO: 9, and is determined by aligning the residues of the two amino acid sequences (i.e., a candidate amino acid sequence and the amino acid sequence of SEQ ID NO: 9) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence is the amino acid sequence being compared to an amino acid sequence present in SEQ ID NO: 9. A candidate amino acid sequence can be isolated from a natural source, or can be produced using recombinant techniques, or chemically or enzymatically synthesized. Preferably, two amino acid sequences are compared using the Blastp program of the BLAST 2 search algorithm, as described by Tatusova et al. (FEMS Microbiol Lett., 174, 247-250 (1999)), and available at www.ncbi.nlm.nih.gov/gorf/bl2.html. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, amino acid identity is referred to as “identities.” SB polypeptides preferably have a molecular weight range of about 35 kDa to about 40 kDa on about a 10% SDS-polyacrylamide gel.

The SB polypeptides useful in some aspects of the invention include an active analog or active fragment of SEQ ID NO: 9. An active analog or active fragment of an SB polypeptide is one that is able to mediate the excision of a transposon from a non-integrating vector, preferably a non-integrating viral vector. An active analog or active fragment can bind the inverted repeat sequences of SEQ ID NOs: 7-8 and direct repeat sequences (SEQ ID NOs: 2-5) from a transposon, as well as a consensus direct repeat sequence (SEQ ID NO: 1).

Active analogs of an SB polypeptide include polypeptides having amino acid substitutions that do not eliminate the ability to excise a transposon from a non-integrating vector. Substitutes for an amino acid may be selected from other members of the class to which the amino acid belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, aspartate, and glutamate. The positively charged (basic) amino acids include arginine, lysine, and histidine. The negatively charged (acidic) amino acids include aspartic acid and glutamic acid. Examples of preferred conservative substitutions include Lys for Arg and vice versa to maintain a positive charge; Glu for Asp and vice versa to maintain a negative charge; Ser for Thr so that a free —OH is maintained; and Gln for Asn to maintain a free NH₂.

Active analogs, as that term is used herein, also include modified polypeptides. Modifications of polypeptides of the invention include chemical and/or enzymatic derivatizations at one or more constituent amino acids, including side chain modifications, backbone modifications, and N- and C- terminal modifications including acetylation, hydroxylation, methylation, amidation, and the attachment of carbohydrate or lipid moieties, cofactors, and the like. Active fragments of a polypeptide include a portion of the polypeptide containing deletions or additions of one or more contiguous or noncontiguous amino acids such that the a resulting polypeptide will excise a transposon from a non-integrating vector.

The coding sequence encoding an SB polypeptide can have the nucleotide sequence of SEQ ID NO: 10, which encodes the amino acid sequence depicted at SEQ ID NO: 9. In addition to the amino acid substitutions discussed above that would necessarily alter the SB-encoding nucleotide sequence, there are other nucleotide sequences encoding an SB polypeptide having the same amino acid sequence as an SB protein such as SEQ ID NO: 9, but which take advantage of the degeneracy of the three letter codons used to specify a particular amino acid. The degeneracy of the genetic code is well known to the art and is therefore considered to be part of this disclosure. Further, a particular nucleotide sequence can be modified to employ the codons preferred for a particular cell type. These changes are known to those of ordinary skill in the art and are therefore considered part of this invention.

Methods of Making the Composition

The present invention is also directed to methods of making a cationic polymer, preferably PEI, that includes a targeting group, preferably a saccharide, bound to an amine, preferably a primary amine, of the cationic polymer. The cationic polymers, preferably PEI, that are used to make the cationic polymers of the present invention preferably have an average molecular weight (MW) within a range defined by a lower limit of about 0.5 kiloDaltons (kDa), more preferably about 10 kDa, and an upper limit of about 800 kiloDaltons (kDa). Preferably, the average molecular weight of the PEI is about 25 kDa. The average molecular weight of PEI can be determined by methods known to the art including gas phase electrophoretic mobility molecular analysis (GEMMA) (Yoon et al., Proc. Natl. Acad. Sci. U.S.A., 93, 2071 (1996)), light scattering, and scanning and transmission electron microscopy (see, for instance, Kren et al., Proc. Natr. Acad. Sci. U.S.A., 96, 10349 (1999)). The upper limit on the MW of the PEI is determined by the toxicity and solubility of the PEI. Toxicity and insolubility of molecular weights greater than about 1.3 megaDaltons (MDa) typically makes such PEI material less suitable for use in the methods described herein.

In one aspect of the invention, the method of making a cationic polymer of the invention includes converting a saccharide to an aldonic acid, and combining the aldonic acid, a cationic polymer and 1-ethyl-3-(dimethylaminopropyl)-carbodiimide under conditions suitable to couple the aldonic acid to primary amines of the cationic polymer. In another aspect, the method includes combining a saccharide, a cationic polymer and 1-ethyl-3-(dimethylaminopropyl)-carbodiimide under conditions to couple the saccharide to primary amines of the cationic polymer.

The conjugation of a targeting group, preferably a polypeptide targeting group, with PEI can also be accomplished by, for instance, modifying the PEI primary amines using the heterobifunctional cross linker, N-succinimidyl 3-(2-pyridyldithio) propionate (SPDP). This reagent reacts with primary amines to provide a 4-carbon spacer with an end 2-pirdyldithiol group. This can then be reacted with dithiothreitol (DTT) to produce a sulfhydryl modified PEI. A similar SPDP activation of the targeting group primary amines is done and the derivatized ligand reacted with the sulfhydryl modified PEI thus attaching the ligand to the PEI via a disulfide linkage. The use of the longer LC-SPDP molecule as the heterobifunctional activating agent permits the conjugation of the targeting groups with increased spacer length. This methodology has the advantage of requiring only one modification of PEI that can then be used to generate the different ligand-PEI conjugates. Moreover, this method of conjugation of other proteins to cationis polymers for delivery of a targeting group does not appear to effect the receptor-mediated uptake of the PEI nor its complexation with target group.

Methods of making a cationic polymer of the invention where the targeting group is covalently bound to the secondary amine are known to the art (see, for instance, Bandyopadhyay et al., J. Biol. Chem., 274, 10163-101172 (1990)), one of which is disclosed herein in the Examples.

Whether a targeting group is bound to a primary amine or a secondary amine of a cationic polymer can be determined using methods known to the art. Typically, the number of moles of free secondary amines in the cationic polymer conjugate, preferably the PEI conjugate, is determined as described in Examples 1 and 2. The number of moles of free primary amines in the cationic polymer conjugate, preferably the PEI conjugate, is determined as described in Examples 1 and 2. Optionally, the total number of amines is also determined as described in Example 1.

A cationic polymer, preferably PEI, that includes a targeting group covalently bound to a primary amine, of the cationic polymer preferably has at least about 1%, more preferably at least about 3%, most preferably at least about 8% of the primary amines derivatized with a targeting group. Preferably, the number of secondary amines of such a cationic polymer, preferably PEI, derivatized with a targeting group is undetectable using the methods described herein. More preferably, the cationic polymer, preferably PEI, has no greater than about 1% of the secondary amines derivatized with a targeting group.

After covalent attachment of a targeting group to a cationic polymer, preferably PEI, the average molecular weight of the PEI:targeting group conjugate is typically less than the average molecular weight of the PEI that was initially used to make it. Preferably, the average molecular weight of a PEI:targeting group conjugate is from about 5 kDa to about 500 kDa more preferably, from about 10 kDa to about 12 kDa.

Optionally and preferably, a cationic polymer:targeting group conjugate also includes a biologically active compound complexed with the conjugate. A molecular complex forms between the negatively charged biologically active compound and the positively charged cationic polymer:targeting group conjugate. The interaction of the two highly charged substrated in non-covalent, and the branched structure of the cationic polymer condenses the DNA so it is a smaller particle. Accordingly, as used herein, the term “complexed with” means there is a non-covalent interaction between the biologically active compound and the cationic polymer. The combination of a cationic polymer:targeting group conjugate and a biologically active compound is referred to herein as a “molecular complex.” The non-covalent interaction may includes, for instance, ionic bonds, hydrogen bonds, and Van der Waals forces. The non-covalent interaction may also be due to steric hinderance, i.e., the biologically active compound is too large to diffuse from the cationic polymer.

Methods for complexing a biologically active compound with a cationic polymer:targeting group conjugate typically include adding a solution including the cationic polymer:targeting group to a solution including the biologically active compound and mixing for about 10 to about 30 seconds. The solution containing the biologically active compound includes water, and preferably contains a carbohydrate monomer, preferably dextrose, at a concentration of from about 4% to about 6%, preferably about 5%. The solution containing the conjugate is typically ultrapure water. Alternatively and preferably, the methods include adding a solution, preferably containing 5% dextrose, including the biologically active compound to a solution including both unconjugated cationic polymer and cationic polymer:targeting group and mixing for about 10 to about 30 seconds. Preferably, unconjugated and the conjugated cationic polymer are the same cationic polymer, for instance, both are PEI.

The ratio of the biologically active compound to the conjugate or, preferably, of the biologically active compound to the amount of unconjugated and conjugated cationic polymer, that is optimal for delivery of the biologically active compound to a cell varies by the type of cell that is targeted. The optimal ratio can be readily determined by one of skill in the art by varying the ratio of the moles of the biologically active compound and the moles of amines of conjugate or of unconjugated and conjugated cationic polymer that are combined to form a molecular complex. Molar ratios of biologically active compound to amines present in the cationic polymer preferably range from about 1 to about 1 (1:1) and about 1 to about 10 (1:10).

When the cationic polymer solution contains both unconjugated and conjugated cationic polymer, the ratio of the two types of cationic polymers that is optimal for delivery of the biologically active compound to a cell varies by the type of cell that is targeted. The optimal ratio can be readily determined by one of skill in the art by varying the ratio of the two types of cationic polymers. The ratio used may be the number of amines in the unconjugated and the number of amines in the conjugated cationic polymers. Molar ratios of unconjugated cationic polymer to conjugated cationic polymer preferably range from about 1:2 to about 2:1.

The compositions of the present invention optionally further include a pharmaceutically acceptable carrier. Typically, the composition includes a pharmaceutically acceptable carrier when the composition is used as described below in “Methods of Use.” The compositions of the present invention may be formulated in pharmaceutical preparations in a variety of forms adapted to the chosen route of administration. Formulations include those suitable for parental administration (for instance intramuscular, intraperitoneal, or intravenous), oral, transdermal, nasal, or aerosol. Dosages of the compositions of the invention are typically from about 0.75 mg/kg up to about 185 mg/kg. Dosages of compositions that include naked polynucleotide (described hereinbelow) are typically from about 0.5 mg/kg up to about 16 mg/kg.

The formulations may be conveniently presented in unit dosage form and may be prepared by methods well known in the art of pharmacy. All methods of preparing a pharmaceutical composition include the step of bringing the active compound (e.g., a molecular complex) into association with a carrier that constitutes one or more accessory ingredients. In general, the formulations are prepared by uniformly and intimately bringing the active compound into association with a liquid carrier, a finely divided solid carrier, or both, and then, if necessary, shaping the product into the desired formulations.

Typically, the compositions of the invention will be administered from about 1 to about 5 times per day. The amount of active ingredient that may be combined with the carrier materials to produce a single dosage form will vary depending upon the subject treated and the particular mode of administration. A typical preparation will contain from about 5% to about 95% active compound (w/w). Preferably, such preparations contain from about 20% to about 80% active compound. The amount of active compound in such therapeutically useful compositions is such that the dosage level will be effective to prevent or suppress the condition the subject has or is at risk for.

Formulations suitable for parenteral administration conveniently comprise a sterile aqueous preparation of the composition, or dispersions of sterile powders that include the composition, which are preferably isotonic with the blood of the recipient. Isotonic agents that can be included in the liquid preparation include sugars, buffers, and sodium chloride. Solutions of the composition can be prepared in water, and optionally mixed with a nontoxic surfactant. Dispersions of the composition can be prepared in water, ethanol, a polyol (such as glycerol, propylene glycol, liquid polyethylene glycols, and the like), vegetable oils, glycerol esters, and mixtures thereof. The ultimate dosage form is sterile, fluid and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the composition, preferably by filter sterilization. Preferred methods for preparing powders include vacuum drying and freeze drying of the sterile injectable solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the composition by the animal over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active compound as a powder or granules, as liposomes containing the active compound, or as a solution or suspension in an aqueous liquor or non-aqueous liquid such as a syrup, an elixir, an emulsion or a draught.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid and the like; a lubricant such as magnesium stearate; a sweetening agent such as sucrose, fructose, lactose or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active compound may be incorporated into sustained-release preparations and devices.

Methods of Use

The present invention further provides methods for delivering a biologically active compound to a vertebrate cell. The method includes introducing to a vertebrate cell a cationic polymer that includes both a targeting group covalently bound to an amine, preferably a primary amine, of the cationic polymer and a biologically active compound complexed with the cationic polymer. The vertebrate cell may be ex vivo or in vivo. As used herein, the term “ex vivo” refers to a cell that has been removed from the body of a subject. Ex vivo cells include, for instance, primary cells (e.g., cells that have recently been removed from a subject and are capable of limited growth in tissue culture medium), and cultured cells (e.g., cells that are capable of extended culture in tissue culture medium). As used herein, the term “in vivo” refers to a cell that is within the body of a subject.

With ex vivo cells, the cationic polymer is typically introduced by adding the cationic polymer directly to the medium. When the cells are in vivo, the cationic polymer can be introduced systemically (for instance, by intravenous injection) or locally (for instance, by direct injection into the target tissue). Preferably, the cationic polymer is introduced systemically, preferably by intravenous injection.

The cell to which the cationic polymer is delivered depends on the nature of the targeting group that is bound to the cationic polymer. As discussed herein, the target molecule interacts with a molecule present on the surface of a cell, e.g., a receptor. By varying what cell the target molecule interacts with, the cationic polymer will be targeted to different cells. Preferably, the target molecule of the cationic polymer interacts with a molecule present on a liver cell, preferably a hepatocyte. In this aspect of the invention, the target molecule may include, for instance, galactose, N-acetylgalactosamine, triantennary galactose, lactose or asialofeutin. Preferably, the target molecule interacts with a liver cell asialoglycoprotein receptor.

As discussed herein, a biologically active compound may be therapeutic or non-therapeutic. The successful in vivo use of a therapeutic biologically active compound is disclosed in Example 4. This Example demonstrates, inter alia, the correction of the UDP-glucuronosyltransferase-1 coding sequence defect in the Gunn rat model of Crigler-Najjar syndrome type I with a coding sequence delivered using a composition of the present invention. The Gunn rat model is an commonly accepted model for human disease (see, for instance, Chowdhury et al., Adv. Vet. Sci. Comp. Med., 37, 149-173 (1993), and Kren et al., Proc. Natl. Acad. Sci. USA, 96, 10349-10354 (1999)).

The successful in vivo use of a non-therapeutic biologically active compound is disclosed in Example 2. The Example demonstrates, inter alia, the use of a non-therapeutic biologically active compound to show the predicted targeting of a composition of the present invention to the liver, and the ability of the transposon to stably integrate in the genomic DNA of the recipient cells.

The present invention is also directed to methods for the introduction of a polynucleotide to a vertebrate cell, where the polynucleotide is naked. As used herein, the term “naked” indicates the polynucleotide that is introduced to the cell is not associated with anything. For instance, a naked polynucleotide is not associated with any delivery vehicle other than the solution in which the polynucleotide is dissolved. In this aspect of the invention, the polynucleotide includes a transposon, or includes a coding sequence encoding a transposase. Alternatively, the polynucleotide includes both a transposon and a coding sequence encoding a transposase. The vertebrate cell can be in an in utero animal, or in an animal.

The present invention is illustrated by the following examples. It is to be understood that the particular examples, materials, amounts, and procedures are to be interpreted broadly in accordance with the scope and spirit of the invention as set forth herein.

### Example 1

### Attachment of Lactose to Polyethyleneimine (PEI)

This example describes the production of lactose-PEI for use in targeting the PEI to liver, specifically to hepatocytes. The lactose was covalently bound to the secondary amine or the primary amine of the PEI.

Lactosylation of the Secondary Amine of PEI

The method used for conjugating oligosaccharides to the secondary amine of 25 kDa PEI (Aldrich Chemical Co., Milwaukee, Wis.) relied on the ability of the cyanoborohydride anion to selectively reduce the imminium salt formed between an amine and an aldehyde of a reducing sugar (G. Gray, Arch. Biochem. Biophys. 163, 426 (1974)). Briefly, a 0.2 molar (M) stock of the monomeric 43 kDa PEI (CH₂CH₂NH) in 0.2 M ammonium acetate, pH 7.6, was prepared as follows. The PEI was transferred to a tare weighed beaker using a glass pipette to spool the sticky material. Sufficient 0.2 M ammonium acetate/ hydroxide buffer, pH 7.6 was added to the beaker to yield a final concentration of 0.2 M monomeric PEI and the material stirred at room temperature until it was fully in solution. For conjugation of the lactose to the PEI amines, 3 milliliters (ml) of the 0.2 M monomeric PEI in 0.2 M ammonium acetate/ hydroxide buffer, pH 7.6, was incubated with 30 milligrams (mg) of lactose and 8 mg of sodium cyanoborohydride (Sigma Chemical Co., St. Louis, Mo.) at 37° C. for 10 days. The stock PEI used for the conjugation as well as 3 ml of the 0.2 M PEI and 30 mg of lactose without sodium cyanoborohydride anion were also incubated at 37° C. for 10 days. The reaction mixture and controls were dialyzed using 10,000 kDa molecular weight cut-off membranes against ultrapure water (obtained from a MILLI-Q Lab Water System, Millipore, Bedford, Mass.) at 4° C. for 48 hours with 2 changes of water per day.

Lactosylation of the Primary Amine of PEI.

The method used for conjugating oligosaccharides to the primary amine of the 25 kDa PEI used conversion of the carbohydrate hapten to aldonic acid (Moore and Link, J. Biol. Chem. 132, 293 (1940)), and subsequent coupling of the derivatized reducing sugar to the primary amines by 1-ethyl-3-(dimethylaminopropyl)-carbodiimideal., Arch. Biochem. Biophys. 175, 661 (1976)). In brief, 0.6 grams (g) of lactonic acid was added to 4 ml of a 0.8 M solution of 25 kDa PEI in ultrapure water adjusted to pH 4.75 with HCl, while rapidly stirring at room temperature. One-half gram of EDAC was dissolved in 0.75 ml of ultrapure water and added drop-wise over a 30 minute period alternating with the drop-wise addition of 0.5 M HCl to maintain pH at 4.75. The pH of the reaction mixture was monitored for another 15 minutes, adding HCl as needed to maintain a pH of 4.75. Once the pH was stabilized, it was left stirring at room temperature for 6 hours, during which the pH of the solution decreased to about 3.2. The reaction was then quenched by addition of 5 ml of 1 M sodium acetate, pH 5.5. The modified PEI was dialyzed using 3,500 kDa molecular weight cut-off membranes against ultrapure water for 48 hours with 2 changes of water per day at 4° C.

Assays to Measure the Amount and Location of Oligosaccharides Conjugated to PEI

The amount of sugar (as galactose) conjugated with PEI was determined by the rescorcinol method (Monsigny et al., Anal. Biochem., 175, 525 (1988)). The amount of sugar is measured as galactose as the glucose moiety attached to the amine group is not mobilized in the assay, thus galactose not lactose is used for generating the standard curve. Briefly, resorcinol (Sigma Chemical Co.) was made to 6 mg/mi in ultrapure water every 30 days and stored at 4° C. in the dark. Analytical grade sulfuric acid (100 ml) was added to 24 ml of ultrapure water to make a 75% solution, cooled to room temperature and stored in the dark at room temperature for up to 3 weeks. Galactose (0.2 mg/ml) was dissolved in ultrapure water to generate a standard curve, which was linear from 4 μg (22.2 nmoles) to 20 μg (111 nmoles). Aliquots of the standard or lactosylated PEI (L-PEI) are diluted to 200 μl in ultrapure water in glass tubes, and then 200 μl of resorcinol (6 mg/ml) and 1 ml of the 75% sulfuric acid were added sequentially to the samples, which were mixed by vortex and heated to 90° C. for 30 minutes. After cooling them in a cold-water bath in the dark for 30 minutes, the optical density of the samples and standards was determined at 430 nanometers (nm). An alternative method for determining amount of sugar (as galactose) conjugated with PEI is the phenol-sulphuric acid (Dubois et al., Anal. Chem. 28, 350 (1956)).

The number of moles of free primary amines in the L-PEI was determined using ninhydrin reagent with leucine as the standard. PEI is composed of primary, secondary and tertiary amines at a ratio of 1:2:1 (Suh et al., Bioorg. Chem., 22, 318 (1994)), thus, each microliter (μl) of a 0.2 M stock of the monomeric PEI contained 200 nanomoles of amines, with 25% or 50 nanomoles primary amines which were detected in the following assay. Leucine (5 mM) dissolved in ultrapure water was used to generate the standard curve, which was linear between 15 and 100 nanomoles. Aliquots of the standard, 0.2 M stock of the monomeric PEI in ultrapure water or lactosylated PEI (L-PEI) were diluted to 90 μl in ultrapure water in 1.5 ml microcentrifuge tubes. To each tube, 10 μl of 1 M HEPES, pH 7.3, was added and mixed by vortex prior to adding 100 μl of ninhydrin reagent (Sigma Chemical Co.). Following vortexing, the samples were heated for 15 minutes at 100° C. and then placed on ice. Ice-cold ultrapure water (300 μl) was added quickly to each tube followed by 500 μl of 100% ethanol. The solutions were mixed by vortex and the optical density determined at 570 nm. The 0.2 M stock of the monomeric PEI in ultrapure water was used to validate the concentration of this sample, which was diluted to generate the standard curves for assaying the secondary and total amine concentration of the L-PEI.

To determine the number of moles of free secondary amines in the L-PEI, a standard curve was formed using a 0.02 M solution of PEI in ultrapure water, which is linear between 50 and 3000 nanomoles of secondary amines. Several aliquots of the stock and L-PEI were diluted to 1 ml using ultrapure water in glass tubes and 50 μl of ninhydrin reagent (Sigma Chemical Co.) was added to each tube. After vortex mixing vigorously for 10 seconds, color development was allowed to proceed in the dark at room temperature for 12 minutes and the optical density determined at 485 nm.

The number of total amines was determined using 2,4,6-trinitrobenzenesulfonic acid (TNBS) (Snyder et al., Anal. Biochem., 64, 284 (1975)). A standard curve is generated using a 4 mM solution of PEI in ultrapure water, which is linear between 40 and 400 nanomoles of amines. Briefly, aliquots of the standard and L-PEI were diluted to I ml using sodium borate buffer, pH 9.3, in glass tubes and vortex mixed. To each sample, 25 μl of a 0.03 M TNBS solution in ultrapure water was added and the mixture was agitated. Following a 30 minute incubation at room temperature in the dark, the optical density was determined at 420 nm.

Using the above assays, it was established that reductive amination using sodium cyanoborohydride anion covalently attached the lactose to the secondary amines while the EDAC conjugation of the aldonic acid derivative of lactose coupled this oligosaccharide only to the primary amines. Both protocols resulted in derivatization of ˜13% of the total amines of the PEI by the disaccharide.

### Example 2

### Targeting of Plasmid DNA to Hepatocytes by Complexing with PEI

This example describes the production of lactose-PEI and complexing the lactose-PEI with plasmid DNA, and the cellular uptake of lactose-PEI/plasmid DNA by a human hepatoma cell line and by hepatocytes in mice injected via the tail vein.

Conjugation of primary Amines to Lactose.

PEI (Aldrich), average MW 25 kDa, was diluted into a reaction buffer containing 0.15 M NaCl and 0.01 M NaH₂PO₄, pH7.2. The PEI solution was brought to pH 7.4 with Glacial Acetic Acid (GAA) and NH₄OH. The PEI was then sterile filtered through a 0.2 μm filter (Fisher) and stored at 4° C. Lactose (Sigma) (200 mg) was mixed with 100 mg EDAC (Sigma) in a Sarstedt 50 ml conical plastic tube and allowed to dry incubate at room temperature for 1=10 minutes. Twenty milliliters of 0.2 M PEI pH 7.4 in reaction buffer was added to the lactose/EDAC and vortexed until completely dissolved. The solution was incubated in a shaking water bath at 37° C. for 2, 4, 6, 8, and 24 hours. At the indicated time points, samples were taken and dialyzed in 4,000 MW dialysis membrane (Sigma) against an ultrapure water gradient of ≧100 volumes at 4° C. The water was replaced with fresh ultrapure water every 4-6 hours and dialyzed for 2-4 days.

Assays.

The conjugation efficiency was assayed as previously described (Bandyopadhyay et al., J. Biol. Chem., 274, 10163-10172 (1999)). Briefly, for primary amines, samples were diluted to 75 nmol to 750 nmol amine and 3 μl or 6 μl was further diluted into 90 μl with ultrapure water. Then, 10 μl 1 M HEPES, pH 7.3 and 100 μl Ninhydrin Reagent (Sigma) was added. The tubes were incubated at 100° C. for 15 minutes, cooled on ice, then 300 μl ultrapure water and 500 μl ethanol were vortexed into the sample. The samples were read at 570 nm on a Beckman Spectrophotometer. The standard used was 3.925 nM L-leucine.

For secondary amines, 3 μl or 6 μl of sample was diluted into 1000 μl of ultrapure water and 50 μl of Ninhydrin Reagent was added. The samples were very briefly vortexed and then incubated in the dark at room temperature for 10-12 minutes. Results were read at 485 nm and the 0.2 M PEI in reaction buffer, pH 7.4 served as a standard.

For carbohydrates, a standard phenol-sugar reducing assay was performed (Dubois et al, Anal. Chem., 28, 350-356 (1956)) with 40 μl to 50 μl of sample diluted into 500 μl of ultrapure water. 50 μl of 80% (weight/weight) phenol was added and vortexed briefly. Next, 2 mls of pure sulfuric acid (Malinkrodt) was added directly to the samples and then incubated in a 37° C. water bath for 10 minutes. The samples were then diluted with 2 ml of ultrapure water and vortexed until homogeneous. After 10 minutes of cooling at room temperature, the samples were read at 490 nm in a quartz cuvette. Galactose in ultrapure water (10 mg/ml) served as a standard. Protein concentrations were quantified using the Bradford method with Bio-Rad's Bradford Reagent (Hercules, Calif., USA) and 20 μl of sample diluted into 800 μl with ultrapure water.

Conjugating Asialofetuin to Primary Amines.

Asialofetuin (Sigma) (350 mg) was mixed with 100 mg EDAC, as done for the lactose conjugation. 20 mls of 0.2 M PEI in reaction buffer (pH 7.4) was added and the sample was inverted to avoid foaming from the asialofetuin. The sample was then incubated as above and samples were taken at 2, 4, 6, 8, and 24 hours. Samples at these time points were dialyzed and assayed as described above.

Plasmids

Control plasmid. The plasmid pGL3 (Promega, Madison, Wis.) encoding the firefly luciferase gene was amplified from DH5α glycerol stocks in LB medium and purified by affinity chromatography on QIAGEN columns (Qiagen, Chatsworth, Calif., USA) according to manufacturer's suggested protocol. The quality of the DNA was determined by UV spectroscopy and agarose gel electrophoresis (1%) with 0.5μg/mL ethidium bromide.

Plasmids for delivery of Sleeping Beauty. Two different constructs have been created from the initial pT/GFP transposon vector. pT/GFP was constructed by digesting pT (Ivies et al. (Cell, 91, 501-510 (1997)) with MscI and StuI and digesting ns-Xs-GM2 (Meng et al., Proc. Natl. Acad. Sci. USA, 94, 6267-6272 (1997)) with hXoI and BglII. The ends of the XeX-GM2 fragment from the vector SP73 were filled with T4 DNA polymerase, and then inserted into the digested pT vector. pT/GFP was modified to incorporate either the gene encoding SB 10 from pSB 10 or pCMVSB 10. The plasmid pSB 10 encodes the Sleeping Beauty transposase and is described in Ivics et al. (Cell, 91, 501-510 (1997)). pCMVSB10 is a plasmid encoding the Sleeping Beauty transposase under control of the CMV promoter.

The first cis SB construct was generated by linearizing the starting plasmid, pT/GFP using the restriction endonuclease AatII (New England Biolabs, Beverly, Mass.). This enzyme cuts at a single site in the plasmid outside of the pT/GFP transposon cassette. The SB cassette to be inserted was excised from the pSB10 plasmid as an EcoRI/ BamHI cassette, and the EcoRI/ BamHI fragment isolated following 1% electrophoresis of the digested pSB10, using a Qiagen gel purification kit (Qiagen, Inc., Chatsworth, Calif.). Both the excised SB cassette and the linearized pT/GFP plasmid were treated with Klenow enzyme and T4 DNA polymerase (New England Biolabs, Beverly, Mass.), respectively, according to the manufacture's suggested protocol. Following the generation of the blunt ends, pT/GFP was then dephosphorelated by treatment calf intestinal phosphatase (New England Biolabs, Beverly, Mass.) and the two blunt ended DNAs ligated at 25° C. using the rapid ligation buffer and T4 DNA ligase from Promega (Madison, Wis.). Following transformation into frozen chemically competent E. coli DH5α, the bacteria were plated on hard Luria agar (HLA) containing 75 μg/ml ampicillin. Individual colonies were picked, grown overnight in Luria broth (LB) containing 75 μg/ml ampicillin and the plasmids isolated. The plasmids were characterized by restriction endonuclease digestion to confirm the insertion of the SB cassette in the AatII site.

The second cis SB construct was generated by linearizing the starting plasmid, pT/GFP using the restriction endonuclease NarI (New England Biolabs, Beverly, Mass.). This enzyme cuts at a single site in the plasmid outside of the pT/GFP transposon cassette. The SB cassette to be inserted was excised from the pCMVSB10 plasmid as a EcoRI/XbaI cassette. The EcoRI/XbaI fragment was isolated following 1% elecrophoresis of the digested pCMVSB10, using a Qiagen gel purification kit (Qiagen, Inc., Chatsworth, Calif.). Both the excised SB cassette and the linearized pT/GFP plasmid were treated with Klenow enzyme, according to the manufacture's suggested protocol. Following the generation of the blunt ends, pT/GFP was then dephosphorelated by treatment shrimp intestinal phosphatase (Roche Molecular Biochemicals, Indianapolis, Ind.) and both the CMVSB cassette and the dephosphorelated pT/GFP were cleaned up using the PCR purification kit from Qiagen, as suggested by the manufacture. The two blunt ended DNAs ligated at 25° C. using the rapid ligation buffer and T4 DNA ligase from Promega (Madison, Wis.). Following transformation into frozen chemically competent E. coli DH5α, the bacteria were plated on hard Luria agar (HLA) containing 75 μg/ml ampicillin. Individual colonies were picked, grown overnight in Luria broth (LB) containing 75 μg/ml ampicillin and the plasmids isolated. The plasmids were characterized by restriction endonuclease digestion to confirm the insertion of the SB cassette in the NarI site.

The two plasmids that were used for the trans delivery were pSB10 and pT/GFP.

Transfections/Cell Lines

Human Hepatoma cells (HuH-7) were cultured in Dulbecco's modified Eagle medium (DMEM) (Life Technologies) supplemented with 10% heat-inactivated fetal bovine serum (FBS) (Atlanta Biologicals, Norcross, Ga., USA) and 1% Penicillin/Streptomycin (Gibco) as previously described (Bandyopadhyay et al., J. Biol Chem., 274, 10163-10172 (1999)). For transfections, cells were plated at 1 ×10⁵ cells/35 mm dishes (Fisher) using 1% trypsin-EDTA (Life Technologies) digest for 20 minutes. Cells were allowed to recover for 24 or 48 hours post-plating prior to transfection.

Primary rat hepatocytes were harvested and cultured as previously described (Bandyopadhyay et al., Biotechniques, 25, 282-292 (1998)).

pGL3 control vector plasmid (Promega) was complexed at ratios of 1/4, 1/6, or 1/10 nmol phosphate/nmol amine with PEI mixes. PEI mixes were composed of dialyzed unconjugated PEI:dialyzed conjugated PEI in ratios of 1:1, 1:1.5, and 1:2. The phosphate measured was the single 5′ phosphate attached to the 3′—OH group of the adjacent base. The PEI complexes were generated by diluting the control pGL3 plasmid in sterile 5% dextrose (Sigma Chemical, Co., St Louis, Mo.), and the solution mixed by tapping the tube vigorously or vortexing. The appropriate amounts of PEI and L-PEI for the specific nmol phosphate/nmol amine and PEI:L-PEI ratio being investigated were then added to the dextrose/DNA solution and the solution mixed by vigorous tapping followed by vortex mixing. The final transfection solution contained 1 μg of pGL3 plasmid complexed with PEI:L-PEI/25 μl.

The pT/GFP, pSB10 and cis pT/GFP / pSB10 plasmids were complexed in the same manner. In brief, the plasmid DNA was diluted in 5% dextrose, and the dextrose/DNA solution mixed by vortex. For the trans delivery of both pSB 10 and pT/GFP, both plasmids were added to the 5% dextrose solution prior to vortex mixing. The required amounts of L-PEI and PEI for the specific nmol phosphate/nmol amine and L-PEI:PEI ratio being investigated were then added to the dextrose/DNA solution and the solution mixed by vigorous tapping followed by vortex mixing.

Transfection solutions were diluted to 1μg DNA/25 μl in 5% dextrose (Sigma), natural pH or 20 mM HEPES (pH 7.3) and 5% dextrose,. Following a rinse with appropriate medium, transfections were done per 35 mm dish by adding the 25 μl of transfection solution to 1 ml of complete medium. Twenty four hours following transfection, 1 ml of complete medium was added to each dish for 48 hour incubations. Cells were harvested either 24 or 48 hours post-transfection. After removal of the medium, the cells were washed 3 times with 1 ×PBS, pH 7.4 and then 200 μl of 1×Reporter Lysis Buffer (Promega, Corp.) was added. Following a 10 minunte incubation at room temperature, the cell lysates were scraped from the dishes and underwent three freeze/thaw cycles with liquid nitrogen and 37° C. water bath, then pelleted at 13,000 rpm in a microfuge. Samples were assayed for luciferase activity using 60 μl of supernatant and 300 μl of luciferase reagent (Promega) in a Berkholdt luminometer for three 20-second reads.

The altered protocol for attaching lactose to the PEI by covalently coupling the sugars to only the primary amines resulted in a surprisingly dramatic improvement in the transfection efficiency of the polycation complex. Luciferase activity 48 hours post-transfection was increased from an average of 1×10⁷ relative light units/mg protein with 2 μg of PGL3 plasmid to at least 7.2×10⁸ units/mg protein using 1 μg of plasmid. Moreover, the modification resulted in significantly reduced nonspecific binding to both isolated hepatocytes as well as in vivo. Markedly increased nuclear labeling of the fluorescein-labeled chimeric ON compared to the secondary amine-modified PEI complexes was also observed.

The ability of primary lactosylated PEI (L-PEI) to function as a transfecting agent was examined using the pGL3 reporter plasmid (Promega, Corp., Madison Wis.), which encodes luciferase. Parallel transfections of HuH-7 human hepatoma cells were performed to establish if the primary PEI would function more effectively as a transfecting agent alone or in combination with unmodified PEI. Several different ratios of unmodified PEI to lactosylated PEI were tried, and it was found that mixing PEI, with L-PEI significantly improved the transfection efficiency. The results using 1 μg of plasmid DNA for each transfection are summarized in Table 1.

TABLE 1
Transfection efficiency and ratio of PEI to PEI
lactosylated at the primary amines.

| Ratio of PEI to L-PEI | Relative light units/mg of protein |
|---|---|
| 0:1 | 1.2 × 10⁷ |
| 1:0 | 3.6 × 10⁵ |
| 1:1 | 1.7 × 10⁸ |
| 1:1.5 | 2.3 × 10⁹ |
| 1.5:1 | 9.0 × 10⁸ |

In Vitro Delivery of pT/GFP and cis pT/GFP/SB using naked DNA. As the negative control for experiments in tissue culture, the cells with the naked plasmid DNAs at the same concentrations were transfected. The DNA was diluted in the transfection vehicle (20 mM HEPES buffered glucose, pH 7.3) and an equivalent amount added to the cultured cells. Significant uptake or expression of either the pT/GFP, pT/GFP+pSB10, or cis pT/GFP/SB was not observed in the cultured cells when delivered as naked DNA.

Competition Experiments

Competition experiments to demonstrate the asialoglycoprotein mediated uptake of the L-PEI:PEI/DNA were performed by adding either asialofetuin, a natural ligand for the receptor or D-galactose to the culture media prior to the addition of the transfection solutions. In brief, 100 μl of asilaofetuin (10 mg/ml) in sterile water or 50 μl of 2 M D-galactose in sterile phosphate buffered saline was added to the 1 ml of culture medium in the 35 mm dish about 5 minutes prior to the addition of the transfecting solution. The cells were then cultured and harvested by rinsing in 1×PBS with lysis buffer as described above.

Confocal Microscopy

To visually assess the degree of plasmid delivery by PEI conjugates, plasmids were labeled with ethidium monoazide bromide (Molecular Probes, Eugene, OR, USA) as previously described (Bandyopadhyay et al., Biotechniques, 25, 282-292 (1998)) and transfected using the PEI ratios above. Cells were fixed 24 hours post-transfection with 4% paraformaldehyde, pH 7.4 and viewed using a MRC1000 confocal microscope (Bio-Rad).

Injection of Adult Mice

The lactosylated PEI/plasmid DNA complexes of pT/GFP, pSB10, or pT/GFP+pSB 10 (trans) or cis pT/GFP/SB were generated as described for the in vitro experiments. They were administered as a single bolus injection via the tail vein in a final volume of 400 μl of 5% dextrose. To demonstrate receptor-mediated uptake of our lactosylated PEI/plasmid DNA complexes, ligand competition experiments were performed in vivo. Mice were injected via tail vein with the cis pT/GFP/SB using lactosylated PEI (10 μg/20 g animal body weight) with or with out a bolus of (10 mg/100 g animal body weight) of asialofetuin, 3 minutes prior to and 3 minutes after the administration of the lactosylated PEI complexed cis pT/GFP/SB. An additional bolus injection of ASF was administered 4 hours after the injection of the lactosylated PEI complexed cis pT/GFP/SB. The use of the asialofetuin, a natural ligand for the asialoglycoprotein receptor, should significantly block the receptor-mediated uptake of the lactosylated PEI complexed cis pT/GFP/SB.

Western Blot Analysis

To isolate total protein, preweighed frozen tissues were subjected to dounce homogenization in 8 volumes of 4° C. buffer A (10 mM Tris, pH 7.6, containing 5 mM MgCl2, 1.5 mM potassium acetate, 2 mM DTT, and 1 tablet of EDTA free mini COMPLETE protease inhibitor (Roche Molecular Biochemicals, Indianapolis, IN)/10 ml) on ice. For fractionation, the homogenized protein solution was centrifuged at 500×g 10 minutes, 4° C., the pellet was washed with Buffer A, and respun as before. The supernatants were pooled and recentrifuged at 5000 rpm for 10 minutes to isolate the cytoplasmic proteins in the supernatant. Nuclear proteins were isolated by washing the initial pellet with buffer B (10 mM Tris, pH 7.6, containing 5 mM MgCl2, 0.25 M sucrose, 0.5% Triton X-100, 1 tablet of EDTA free mini COMPLETE protease/10 ml., followed by two centrifugations (500×g for 5 minutes each) and resuspension cycles, and finally sonciated twice at 4° C. using a microsonicator. Samples were spun again at 500×g and the supernatant containing the nuclear proteins saved. All proteins were quantified using BioRad Bradford protein reagent (BioRad Laboratories, Hercules, Calif.) as suggested by the manufacturer. Proteins were aliquoted, flash-frozen in liquid nitrogen, and stored at −80° C. To visualize the GFP protein (approximately 27.5 kDa), 15% polyacylamide gels were prepared as described (Trembley et al., Cell Growth & Differ., 7, 903-916 (1996)) and 100 μg of protein was loaded per lane. As a positive control, 25 μg of total protein extract from a viral-transduced GFP cell line was run and Rainbow Markers (Amersham, Pharmicia Biotech, Piscataway, N.J.) were used as molecular weight standards. The PAGE was performed at 18 mAmps and the gels transferred to nitrocellulose membrane (Amersham, Pharmicia Biotech) using a TRANSBLOT (BioRad Laboratories) as suggested by the manufacturer. The membranes were then treated with 15% hydrogen peroxide for 15 to 30min rocking at room temperature, then blocked with 5% milk in 1×TBS, pH 7.4 for 2 hours at room temperature. Membranes were incubated overnight at 4° C. rocking with either a primary polyclonal Rabbit IgG Anti-GFP (LIVING COLORS, Clontech) or a mouse monoclonal anti-GFP (B-2) (Santa Cruz Biotechnology, Inc., Santa Cruz, Calif.) in 5% milk at a dilution of 1:6000, and 1:300 respectively. Membranes were rinsed with 1×TBS+0.2% Tween-20 for 5 minutes three times and then incubated for 2 hours at room temperature with secondary Goat IgG Anti-Rabbit, or secondary Goat IgG Anti-Mouse (BioRad Laboratories) in 5% milk at a dilution of 1:5000. Membranes were rinsed as indicated above and the proteins detected by Chemiluminescence (Amersham, Pharmicia Biotech) as suggested by the manufacturer.

Results

Previous reports have shown size analysis by dynamic laser light scattering of complexed PEI/DNA particles using secondary saccharide-PEI demonstrated that PEI could condense a plasmid molecule to about 100 nm (Erbacher et al., J. Gene Med., 1, 210-222 (1999)). It was hypothesized that condensation could be improved upon by placing the lactose moiety on a more external face of the complex, such as a primary amine of the PEI. This would not only decrease the overall size of the particle, but would possibly increase the targeting and cellular uptake efficiency and efficacy. Using 25 kDa PEI (Aldrich), anhydrous D-lactose was reacted in a sodium phosphate/sodium chloride buffer for 8 hours at 42° C. using EDAC to catalyze the reaction. Following dialysis, the conjugation by primary and secondary amine assays was assayed and phenol-sugar assays. On average, 3% to 8% of the primary amines of the 25 kDa PEI were conjugated. It also appeared that the reaction broke the 25 kDa PEI polymer into oligomers around 10 kDa to 12 kDa in size. This size reduction of the PEI was unexpected and was confirmed by dialysis studies, using membranes of either 10,000 kDa molecular weigh cut off (MWCO) or 3,000 kDa (MWCO). Interestingly, the smaller size of the L-PEI polymers decreased the toxicity of this molecule significantly, and improved the transfection efficiency relative to the PEI derivitized with lactose on the secondary amines.

To test the efficacy of this new conjugate at delivering plasmids to a hepatic target, human hepatoma cells (HuH-7), primary rat hepatocytes (1 HEPS), and immortalized human hepatocytes (MIHA) were transfected with 0.25 μg, 0.5 μg, 1.0 μg, or 2.0 μg of pGL3 luciferase control vector (Promega). This vector was ionically complexed to the primary Lac-PEI at ratios of 1:2, 1:4, 1:6, and 1:10 DNA phosphates: total PEI amines, in mixtures of unconjugated PEI:primary Lac-PEI of 1:1, 1:1.5, and 1:2. Total volumes were based on 1.0 μg=50 μl , with 5% native pH dextrose acting as the carrier solution. Transfection solutions were added dropwise to 35 mm dishes containing 2×10⁵ cells in 1 ml of serum-containing culture media. Transfections were harvested as specified by manufacturer (Promega) for luciferase assay at 24 hours, or supplemented with 1 ml of appropriate medium and harvested at 48 hour or 72 hour time points. Luciferase and Bradford protein assays were performed as suggested by the manufacturer using 60 μl protein extract and 300 μl Promega Luciferase Reagent; and 20 μl protein extract and 200 μl BioRad Reagent, respectively.

With the success of the new delivery system, the ability to target plasmids to cells in vivo was tested. Previous experiments (Ivics, et al, Cell, 91, 501-510 (1997)) have shown that Sleeping Beauty transposons can transpose HeLa cells in vitro using calcium phosphate transfection methods and mouse hepatocytes in vivo using the hydrodynamic-push method (Yant et al, Nat. Genet., 25, 35-41 (2000)). As the delivery potential of SB transposons has great relevance to mammalian and specifically human diseases, plasmids containing a GFP transposon with (cis) and without (trans) the SB transposase were designed. In designing the cis plasmid, the transposase was placed outside of the inverted repeat/direct repeat (IR/DR) borders so as to not recreate an autonomous transposon. The same transfection protocol was repeated, fixing the cell dishes with 4% paraformaldehyde in 1×PBS at 30 minutes, 2 hours, 4 hours, 8 hours, 16 hours, 24 hours, 48 hours, 72 hours, and 120 hours post-tranfection as described (Bandyopadhyay et al., J. Biol. Chem., 274, 10163-10172 (1999).

Confocal microscopic analysis of the transfected cells demonstrated that not only does the primary Lac-PEI effectively transfect these cell lines, but also cells transfected with transposons and SB transposase display GFP activity for extended times than those dishes with the transposon alone. These results indicated that the GFP gene had successfully integrated into the genome and was not lost through plasmid degradation.

Next, the efficacy of the primary Lac-PEI at delivering these GFP transposons to liver tissue in vivo was determined. Wildtype B6 or B6+/gus mice were injected via tail vein with primary Lac-PEI complexed to the transposon encoding plasmids in the same 5% native pH dextrose carrier solution used for the tissue culture studies. From the tissue culture experiments, 1:6 DNA phosphates:total PEI amines with a mix of 1:1.5 unconjugated PEI: primary Lac-PEI were identified to be the optimal delivery mixture. Initial injections were given at 10 μg and 50 μg of DNA; later the dose was lowered to 5 μg of DNA for each 10 g of mouse body weight, as the mice injected with the cis construct at 50 μg and half of the mice at 10 μg in both the cis and trans designs died. Mice were injected with: dextrose only, dextrose+PEI, dextrose+PEI+transposon alone, dextrose+PEI+transposase alone, dextrose+PEI+trans design, and dextrose+PEI+cis design. The mice were fed standard chow and sacked at the following time point: 6 hours, 8 hours, 24 hours, 1 week, 2 weeks, and 8 weeks post-injection. Mutant gus/gus mice, models for the mucopolysaccharidosis type 7 disease, were injected as above, fed Teklad high protein, low fat and antibiotics 3/7 days per week, and sacked at the time points above. All mice were exsanguanated under ether anethesia. Organs were removed, wrapped in foil, flash-frozen in liquid nitrogen, and stored at −80° C. Tissues were sectioned using a cryostat (−20° C. to −30° C.) to a thickness of 10 μm with a 16 mm steel blade. Sections were pressed onto SUPRAFROST PLUS slides (Fisher) and placed on a 37° C. heatblock to dry. Tissues were then fixed with 4% paraformaldehyde for 10 minutes and rinsed with 1×PBS, pH 7.4. Slides were post-fixed with SlowFade/Antifade medium (Molecular Probes) in PBS as suggested by the manufacturer and cover slips were applied. To avoid autofluorscence from the tissues, the samples were viewed within 8 hours of sectioning.

The sectioned tissues were examined by confocal microscopy and indicated that the primary Lac-PEI was effective in targeting PEI:plasmid DNA complexes to the hepatocytes. In fact, the delivery appeared to be quite liver specific as none of the other tissues examined had detectable GFP expression through out the time period investigated. Furthermore, the tissues from animals that received only the pT/GFP transposon exhibited gradually diminishing GFP activity, while the samples from animals that received both transposon and transposase maintained significant GFP expression even 8-weeks after injection. An unexpected and intriguing finding was that the cis delivery of the transposon and transposase resulted in an even distribution of GFP expression throughout the liver. In contrast, the trans delivery of the transposon and transposase displayed a “chunky” GFP expression with patches of very highly expressed GFP adjacent to tissue exhibiting little or non-detectable GFP expression.

To confirm that the Lac-PEI was targeting the liver through receptor-mediated endocytosis, competition experiments were performed in tissue culture using the luciferase reporter plasmid, and in mice using GFP expression. For the tissue culture experiments, 100 nmol D-galactose was added to each 35 mm culture dish, incubated for 5 minutes, and then the Lac-PEI/DNA solution was added as above. The cells were cultured as before, harvesting at 24 hours and 48 hours and the luciferase and protein assays were performed. The luciferase activity in the competition dishes was almost completely diminished, supporting an uptake via receptor-mediated endocytosis. For the in vivo experiment, mice were injected via tail vein with 400 μl of 25 mg/ml asialofetuin (ASF), followed by the Lac-PEI/DNA complexed solution in 400 μl, and another injection of ASF. Four hours later, another injection of ASF was administered to the mice. The use of the asialofetuin, a natural ligand for the asialoglycoprotein receptor, should significantly block the asialoglycoprotein receptor-mediated uptake of the lactosylated PEI complexed cis pT/GFP/SB. Confocal microscopy of the sectioned tissues indicated significant inhibition of uptake/expression (>80%) of the cis pT/GFP/SB in the liver of animals coadministered the asialofetuin. These animals exhibited significant uptake and expression of the cis pT/GFP/SB in lung, heart, kidney and spleen. In contrast, the animals that received only the cis pT/GFP/SB exhibited excellent uptake/expression of GFP in the liver, with little expression of GFP in lung, heart, kidney or spleen. In both groups of animals no detectable GFP expression was observed in the gonads.

To further confirm the presence of GFP in the liver tissues, Western blot analysis was performed using protein extracts isolated from tissue samples. The results of the immunoblots analysis confirmed the confocal microscopic expression patterns observed for pT/GFP, pT/GFP+pSB 10 and cis pT/GFP/SB during the time period followed. The liver tissue from all the mice receiving the pT/GFP transposon with or without the transposase expressed GFP protein through two weeks. By eight weeks post-injection, GFP expression was only observed in the liver of animals that had also received SB transposase in either the cis or trans configuration. This rapid drop in expression from the transposon constructs is consistent with the previously reported human alpha-1 antitrypsin (PTAAT) and Factor IX transposons from Yant et al (Yant et al, Nat. Genet., 25, 35-41 (2000)). The high levels of GFP expression may be lethal to cells and the deaths of these high producing cells leads to a dilution effect of the GFP protein expression and a more sudden decay curve.

To determine is the GFP activity was due to episomal expression, Southern Blot analysis was performed using genomic DNA isolated from frozen livers. The liver DNA was isolated from animals that had received either pT/GFP alone or pT/GFP+SB transposase in either the cis or trans configuration using Qiagen genomic DNA isolation tips according to the manufacture's specifications. The genomic DNA was then digested with AatI, and 10 μg of the genomic DNA subjected to electrophoresis on a 1% agarose gel, and transferred to Gene Screen Plus (BioRad Laboratories) as previously described (Kren et al., Am. J. Physiol., 270, G763-G777 (1996)). A 750 bp fragment of the β-lactamase gene encoding the plasmid borne ampicillin resistance was labeled with ³²p and used as a hybridization probe. The Southern blot was hybridized, washed and the bands detected by autoradiography as previously described (Kren et al., Am. J. Physiol., 270, G763-G777 (1996)). The autoradiograms confirmed that at 1 week, the liver DNA from animals that had received either pT/GFP alone or pT/GFP+SB transposase in either the cis or trans configuration all had episomal plasmid still present. In contrast, the liver DNA from the animals sacrificed eight weeks post-injection exhibited no episomal plasmid presence. This confirmed that the GFP protein expression in the livers from the animals that received both the pT/GFP transposon and SB transposase eight weeks post-injection was not due to episomal plasmid expression. This data also suggests that the GFP protein expression observed in the liver of the animals that received the transposon alone at one and two weeks was most likely due to episomal plasmid expression. The Southern blot was then probed with a ³²p labeled 757 bp fragment corresponding to the GFP coding sequence in the transposon. The limits of detection of the Southern blot analysis were also established using genomic DNA spiked with known concentrations of plasmid DNA. The detection limit of 10 copies of plasmid per cell as determined by the spiked plasmid analysis, precluded the detection of unique transposon integration sites in the genomic DNA. However, a heavier background smear of radioactivity was observed in the 8-week genomic DNA lanes from the animals that received SB transposase in addition to the GFP transposon. This strongly suggested that the GFP expression seen in both the confocal microscopy and the Western blots is due to transposed GFP genes.

In summary, through both in vitro and in vivo experiments, these data indicate that primary Lac-PEI is a more effective transfection agent than other PEI conjugates such as secondary Lac-PEI. This form of PEI appears far more efficacous and less toxic than the 800 kDa PEI conjugated to lactose at the secondary amine and 25 kDa PEI conjugated to lactose at the secondary amine at delivery of DNA to cells both in tissue culture and through in vivo cell targeting. These data also demonstrate the combination of this targeting system with the Sleeping Beauty transposon can deliver large scale genetic material to the mammalian genome at rates more than double previous reports (Yant et al, Nat. Genet., 25, 35-41 (2000)). Future experiments are aimed at proving the ability of the SB system to induce clinically significant, permanent changes in disease models, as well as site-directed transposon integration. Preliminary experiments show that GFP transposons can be effectively targeted to livers of mucopolysaccharidosis disease models, at levels suggestive of clinical significance.

Example 3

### In Utero Delivery of the Sleeping Beauty transposon

In the first set of experiments, mice at day 14/15 of gestation were injected in utero with 0.5 μg the pT/GFP with SB supplied in either the cis or trans configuration or 4.0 μg of pT/GFP alone (control) in a final volume of 5 μl of sterile ultrapure water as previously described (Blazar et al., Blood, 85, 4353-4366 (1995)). PEI was not used. Fluorescent confocal microscopy was done at 1, 8 and 12 weeks after birth, and indicated that GFP expression was observed post-birth only in those fetuses injected with catalytically active transposase, in either the cis or trans configuration. Major sites of GFP expression by confocal microscopy were liver, spleen, small intestine, and kidney. At 1 week, significant GFP expression was also detected in the heart, bone, muscle, and lung.

Western Blot analysis of GFP expression in liver and spleen from animals injected in utero with 0.5 μg the pT/GFP with SB supplied in either the cis or trans configuration or 4.0 μg of pT/GFP alone (pT/GFP) was done. The total protein was isolated and the western blots processed as described above. The immunoblots were incubated overnight at 4° C. rocking with either a primary polyclonal Rabbit IgG Anti-GFP (LIVING COLORS, Clontech, ) or a mouse monoclonal anti-GFP (B-2) (Santa Cruz Biotechnology, Inc., Santa Cruz, Calif.) in 5% milk at a dilution of 1:6000, and 1/300 respectively. Membranes were rinsed with and then incubated for 2 hours at room temperature with secondary Goat IgG Anti-Rabbit, or secondary Goat IgG Anti-Mouse (BioRad Laoboratories) and the proteins detected by Chemiluminescence (Amersham, Pharmicia Biotech) as described by the manufacturer. The results indicated that in liver tissue from animals 8 and 12 weeks post-birth both cis and trans delivery systems for SB resulted in low- to mid-level expression of GFP, while 4 μg of GFP transposon construct alone resulted in no detectable expression. In the 8 and 12 week post-birth animals, cis delivery of the catalytically active SB transposase resulted in dose-dependent GFP expression in the liver by both confocal and western blot analysis. In contrast, trans delivery of the transposon system was independent of dose, and resulted in varied GFP expression, albeit with good correlation between confocal microscopy and western blot analysis.

In the second set of experiments, animals were injected via tail vein with 5 μg of the pT/GFP with SB supplied in either the cis (pT/GFP/SB) or trans configuration (pT/GFP+pSB10), or 5 μg of pT/GFP alone, or the delivery system alone (L-PEI). Animals were sacrificed 8 weeks later and the GFP expression in liver determined by confocal microscopy.

Western Blot analysis of GFP expression in liver from animals injected via tail vein with 5 μg the pT/GFP with SB supplied in either the cis (pT/GFP/SB) or trans configuration (pT/GFP+pSB 10), or 5 μg of pT/GFP alone, or the delivery system alone (L-PEI) or the vehicle alone (Dextrose). Animals were sacrificed 1, 2 or 8 weeks later and the GFP expression in liver determined using 2 different primary antibodies to GFP. Only animals receiving SB in either the cis or trans configuration expressed GFP by either confocal microscopy or western blot analysis.

### Example 4

### Correction of the UDP-glucurosyltransferase Gene Defect in the Gunn Rat Model of Crigler-Najjar Syndrome Type I

In the animal model of Crigler-Najjar syndrome type I, rats have a deficiency of bilirubin UDP-glucuronosyltransferase-I (UGT1A1). A plasmid encoding human UGT1A1 under control of an EF1αpromoter (pTUGT1A1) flanked by the IRDRs was constructed. A cis construct was also generated containing the catalytically active transpsoase under control of the CMV promoter on the same plasmid. Preliminary results following tail vein administration of the L-PEI cis pTUGT1A1 construct indicate that it is effective in lowering the serum bilirubin levels in vivo.

The complete disclosure of all patents, patent applications, and publications, and electronically available material (e.g., GenBank amino acid and nucleotide sequence submissions, and computer programs) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A cationic polymer comprising a primary amine and a targeting group covalently bound to the primary amine, wherein the targeting group targets a cell of interest by interacting with the surface of the cell.

2. A cationic polymer comprising a primary amine and a targeting group covalently bound to the primary amine, wherein the targeting group targets a cell of interest by interacting with the surface of the cell and wherein the cationic polymer comprises a polyethyleneimine.

3. A polyethyleneimine comprising a targeting group covalently bound to a primary amine, wherein the targeting group comprises a targeting group that targets a liver cell.

4. A polyethyleneimine comprising a targeting group covalently bound to a primary amine, wherein the targeting group comprises a lactose.

5. A molecular complex comprising:
a polyethyleneimine comprising a targeting group covalently bound to a primary amine; and
a biologically active compound.

6. A molecular complex comprising:
a polyethyleneimine comprising a targeting group covalently bound to a primary amine; and
a biologically active compound comprising a polynucleotide.

7. A molecular complex comprising:
a polyethyleneimine comprising a covalently bound targeting group; and
a polynucleotide comprising a nucleic acid sequence flanked by inverted repeat sequences that bind a transposase.

8. A molecular complex comprising:
a polyethyleneimine comprising a targeting group covalently bound to a primary amine; and
a polynucleotide comprising a nucleic acid sequence flanked by inverted repeat sequences that bind a transposase.

9. A molecular complex comprising:
a polyethyleneimine comprising a covalently bound targeting group;
a first polynucleotide comprising a nucleic acid sequence flanked by inverted repeat sequences that bind a transposase; and
a second polynucleotide comprising a coding sequence encoding a transposase that binds to the inverted repeat sequences.

10. A molecular complex comprising:
a polyethyleneimine comprising a targeting group covalently bound to a primary amine;
a first polynucleotide comprising a nucleic acid sequence flanked by inverted repeat sequences that bind a transposase; and
a second polynucleotide comprising a coding sequence encoding a transposase that binds to the inverted repeat sequences.

11. A molecular complex comprising:
a polyethyleneimine comprising a covalently bound targeting group; and
a polynucleotide comprising a nucleic acid sequence flanked by inverted repeat sequences that bind a transposase and a coding sequence encoding a transposase that binds to the inverted repeat sequences.

12. A molecular complex comprising:
a polyethyleneimine comprising a targeting group covalently bound to a primary amine; and
a polynucleotide comprising a nucleic acid sequence flanked by inverted repeat sequences that bind a transposase and a coding sequence encoding a transposase that binds to the inverted repeat sequences.

13. A method for making a cationic polymer:targeting group conjugate comprising:
converting a lactose to an aldonic acid; and
combining the aldonic acid, a polyethyleneimine and 1-ethyl-3-(dimethylaminopropyl)-carbodiimide under conditions suitable for coupling the aldonic acid to primary amines of the polyethyleneimine to yield the cationic polymer:targeting group conjugate.

14. A method for making a cationic polymer:targeting group conjugate comprising combining a lactose, a polyethyleneimine, and 1-ethyl-3-(dimethylaminopropyl)-carbodiimide under conditions suitable for coupling the lactose to primary amines of the polyethyleneimine to yield the cationic polymer:targeting group conjugate.

15. A composition comprising the cationic polymer of claim 1 and a pharmaceutical carrier.

16. A pharmaceutical composition comprising a molecular complex comprising:
a polyethyleneimine comprising a covalently bound targeting group; and
a polynucleotide comprising a nucleic acid sequence flanked by inverted repeat sequences that bind a transposase and a coding sequence encoding a transposase that binds to the inverted repeat sequences, wherein the nucleic acid sequence comprises a coding sequence encoding bilirubin UDP-glucuronosyltransferase-1 (UGT1Al).

17. A pharmaceutical composition comprising a molecular complex comprising:
a polyethyleneimine comprising a covalently bound targeting group; and
a first polynucleotide comprising a nucleic acid sequence flanked by inverted repeat sequences that bind a transposase, wherein the nucleic acid sequence comprises a coding sequence encoding bilirubin UDP-glucuronosyltransferase-1 (UGT1A1); and
a second polynucleotide comprising a coding sequence encoding a transposase that binds to the inverted repeat sequences.

18. A method for delivering a biologically active compound to a vertebrate cell, the method comprising introducing into the vertebrate cell a molecular complex comprising:
a cationic polymer comprising a primary amine and a targeting group covalently bound to the primary amine, wherein the targeting group targets a cell of interest by interacting with the surface of the cell; and
a biologically active compound.

19. A method for delivering a biologically active compound to a vertebrate cell, the method comprising introducing into the vertebrate cell a molecular complex comprising:
a polyethyleneimine comprising a lactose covalently bound to a primary amine of the polyethyleneimine; and
a biologically active polynucleotide.

20. A method for delivering a biologically active compound to a vertebrate cell, the method comprising introducing into the vertebrate cell a molecular complex comprising:
a polyethyleneimine comprising a lactose covalently bound to a primary amine;
a first polynucleotide comprising a nucleic acid sequence flanked by inverted repeat sequences that bind a transposase; and
a second polynucleotide comprising a coding sequence encoding a transposase that binds to the inverted repeat sequences.

21. A method for delivering a biologically active compound to a vertebrate cell, the method comprising introducing into the vertebrate cell a molecular complex comprising:
a polyethyleneimine comprising a lactose covalently bound to a primary amine;
a polynucleotide comprising a nucleic acid sequence flanked by inverted repeat sequences that bind a transposase and a coding sequence encoding a transposase that binds to the inverted repeat sequences.

22. A method for delivering a biologically active compound to a vertebrate cell, the method comprising introducing into the vertebrate cell a molecular complex comprising:
a polyethyleneimine comprising a covalently bound lactose;
a first polynucleotide comprising a nucleic acid sequence flanked by inverted repeat sequences that bind a transposase; and
a second polynucleotide comprising a coding sequence encoding a transposase that binds to the inverted repeat sequences.

23. A method for delivering a biologically active compound to a hepatocyte, the method comprising introducing into the hepatocyte a molecular complex comprising:
a polyethyleneimine comprising a covalently bound lactose; and
a polynucleotide comprising a nucleic acid sequence flanked by inverted repeat sequences that bind a transposase.

24. A method for delivering a biologically active compound to a hepatocyte, the method comprising introducing into the hepatocyte a molecular complex comprising:
a polyethyleneimine comprising a covalently bound lactose; and
a polynucleotide comprising a nucleic acid sequence flanked by inverted repeat sequences that bind a transposase, wherein the nucleic acid sequence comprises a coding sequence encoding bilirubin UDP-glucuronosyltransferase-1 (UGT1A1).

25. A method for delivering a biologically active compound to a vertebrate cell, the method comprising introducing into the vertebrate cell a molecular complex comprising:
a polyethyleneimine comprising a covalently bound lactose;
a polynucleotide comprising a nucleic acid sequence flanked by inverted repeat sequences that bind a transposase and a coding sequence encoding a transposase that binds to the inverted repeat sequences.

26. A method for delivering a biologically active compound to a vertebrate cell, the method comprising introducing into a vertebrate cell a molecular complex comprising:
a polyethyleneimine comprising a lactose covalently bound to a primary amine of the polyethyleneimine; and
a biologically active compound.

27. A method for delivering a biologically active compound to a vertebrate cell, the method comprising introducing into a vertebrate cell a molecular complex comprising:
a polyethyleneimine comprising a lactose covalently bound to a primary amine of the polyethyleneimine; and
a biologically active polynucleotide.

28. A method for delivering a biologically active compound to a vertebrate cell, the method comprising introducing to a vertebrate cell in vivo a molecular complex comprising:
a cationic polymer comprising a covalently bound lactose;
a first polynucleotide comprising a polynucleotide flanked by inverted repeat sequences that bind a transposase; and
a second polynucleotide comprising a coding sequence encoding a transposase that binds the inverted repeat sequences.

29. A method for delivering a biologically active compound to a vertebrate cell, the method comprising introducing to a vertebrate cell in vivo a molecular complex comprising:
a cationic polymer comprising a lactose covalently bound to a primary amine of the cationic polymer;
a first polynucleotide comprising a polynucleotide flanked by inverted repeat sequences that bind a transposase; and
a second polynucleotide comprising a coding sequence encoding a transposase that binds the inverted repeat sequences.

30. A method for delivering a biologically active compound to a vertebrate cell, the method comprising introducing to a vertebrate cell a naked polynucleotide comprising a nucleic acid sequence flanked by inverted repeat sequences that bind a transposase, wherein the vertebrate cell is in an in utero animal.

31. A method for delivering a biologically active compound to a vertebrate cell, the method comprising introducing to a vertebrate cell a naked polynucleotide comprising a nucleic acid sequence flanked by inverted repeat sequences that bind a transposase, wherein the vertebrate cell is in an animal.
